(19) United States
(12) Patent Application Publication
(10) Pub. No.: US 2024/0209400 A1
(43) Pub. Date: Jun. 27, 2024

(54) PROCESS FOR PRODUCING FORMATE USING OXYGEN-TOLERANT ENZYMES

(71) Applicant: GWANGJU INSTITUTE OF SCIENCE AND TECHNOLOGY, Gwangju (KR)

(72) Inventors: IN CHAN KWON, Gwangju (KR); JAE HYUN CHA, Gwangju (KR)

(21) Appl. No.: 18/391,941

(22) Filed: Dec. 21, 2023

(30) Foreign Application Priority Data
Dec. 21, 2022 (KR) 10-2022-0180706
Dec. 7, 2023 (KR) 10-2023-0177033

(51) Int. Cl. C12P 7/40; C12N 9/02

(52) U.S. Cl. CPC C12P 7/40; C12N 9/0067; C12N 9/0093; C12Y 112/99006; C12Y 117/01

(57) ABSTRACT

In a process for producing formate, a mixed enzyme by mixing hydrogenase (H₂ase) with oxygen tolerance and formate dehydrogenase (FDH) with oxygen tolerance is prepared, and the mixed enzyme and a gas including H₂, CO₂ and NAD⁺ are mixed such that formate may be produced even in the presence of oxygen, and thereby utilizing hydrogen sources including oxygen, such as coke oven gas.

### CROSS-REFERENCE TO RELATED APPLICATION AND CLAIM OF PRIORITY

This application claims the benefit under 35 USC § 119 of Korean Patent Application Nos. 10-2022-0180705, filed on Dec. 21, 2022, and 10-2023-0177033, filed on Dec. 7, 2023, in the Korean Intellectual Property Office, the entire disclosure of which are incorporated herein by reference for all purposes.

### 1. FIELD OF THE INVENTION

The present invention relates to a process for producing formate.

### 2. DESCRIPTION OF THE RELATED ART

The development of renewable energy technologies to replace fossil fuels is essential for the sustainable growth of the economy and society. Hydrogen (H₂), obtained from various sources such as solar heat, algae, biomass, and by-product gas, is expected to be an alternative fuel with high gravimetric energy density and net-zero carbon dioxide (CO₂) production. However, due to the low volumetric energy capacity of H₂, its transportation and storage as a fuel are limited. Therefore, converting H₂ into a chemical with a high volumetric energy capacity while maintaining the molar energy capacity is required for the commercialization of alternative energy.

Suitable materials for converting H₂ energy should satisfy the following conditions: 1) high energy/volume capacity; 2) low energy loss during the conversion process; 3) liquid material at ambient pressure and temperature; and 4) non-flammable chemicals for safety. Formate satisfies these conditions, such that the conversion of H₂ and CO₂ into formate may be an appropriate approach for the commercialization of alternative energy. The oxidation of H₂ and reduction of CO₂ may occur in hydrogenase (H₂ase) and formate dehydrogenase (FDH), respectively, among oxidoreductases. However, synthetic catalysts for these reactions cannot be applied to various H₂ sources due to their low selectivity, low efficiency, and requirement for precious metals. In particular, cheap and sustainable H₂ sources, such as coke oven gas generated from steel industries, contain a small portion of O₂ (0.0.4 to 1.7%). Because of the transition metal active sites and low potential electrons, most H₂ases and FDHs are inhibited or irreversibly damaged by a trace amount of O₂, thereby limiting the application of H₂ in conversion into formate from various hydrogen sources.

### SUMMARY

An aspect of the present invention is to provide a process for producing formate using oxygen-tolerant enzymes.

Another aspect of the present invention is to provide a process for producing formate utilising a hydrogen source including oxygen.

To achieve the above aspects, the following technical solutions are adopted in the present invention.
1. A process for producing formate including: preparing a mixed enzyme by mixing hydrogenase (H₂ase) with oxygen tolerance and formate dehydrogenase (FDH) with oxygen tolerance; and mixing the mixed enzyme and a gas including H₂, CO₂ and nicotinamide adenine dinucleotide (NAD⁺).
2. The process for producing formate according to the above 1, wherein the gas including H₂ contains O₂.
3. The process for producing formate according to the above 1, wherein the gas including H₂ is obtained from any one source selected from the group consisting of byproduct hydrogen incidentally generated in processes such as a petrochemical or steel industry, gas derived from plastic or solid waste, cokes, naphtha, volcanic gas, mineral water, coal gas, solar heat, algae emissions, biomass, natural gas, fossil fuel, coal, peat, petroleum and natural gasoline.
4. The process for producing formate according to the above 1, wherein the hydrogenase is derived from any one strain selected from the group consisting of Ralstonia eutropha, Escherichia coli and Aquifex aeolicus.
5. The process for producing formate according to the above 1, wherein the formate dehydrogenase is derived from any one strain selected from the group consisting of Rhodobacter capsulatus, Desulfovibrio vulgaris, Clostridium carboxidivorans and Methylobacterium extorquens.
6. The process for producing formate according to the above 1, wherein the hydrogenase includes the amino acid sequence of any of SEQ ID NO: 1 to SEQ ID NO: 5.
7. The process for producing formate according to the above 1, wherein the formate dehydrogenase includes the amino acid sequence of any of SEQ ID NO: 6 to SEQ ID NO: 8.
8. The process for producing formate according to the above 2, wherein the gas including H₂ contains 5% or less of O₂.
9. The process for producing formate according to the above 2, wherein the gas including H₂ contains 2% or less of O₂.
10. The process for producing formate according to the above 1, wherein the hydrogenase and the formate dehydrogenase are mixed in a ratio of 5 to 25:1.

The production process of the present invention enables the development of renewable energy to replace fossil fuels by converting H₂ into formate with a high volumetric energy capacity while maintaining the molar energy capacity.

The production process of the present invention enables the production of formate even in the presence of oxygen.

The production process of the present invention enables the production of formate using aerobic microorganisms or oxygen-tolerant enzymes derived from the microorganisms.

The production process of the present invention uses enzymes, thereby making it possible to produce H₂ and CO₂ into formate without by-products.

The present invention manufactures formate using enzymes, thereby enabling the reuse of the enzymes without loss of activity even after the production of the formate.

The production process of the present invention utilizes the hydrogen (H₂) source including oxygen, thereby making it possible to produce formate.

The production process of the present invention uses hydrogenases and formate dehydrogenases, thereby making it possible to produce formate from the hydrogen source including oxygen.

The production process of the present invention may adjust a ratio of hydrogenase and formate dehydrogenase, thus to easily increase the production of formate.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic view showing the production process of formate through an NAD⁺-dependent cascade reaction of ReSH and RcFDH in the presence of O₂;

FIGS. 2A and 2B show SDS-PAGE of purified proteins ReSH and RcFDH stained with Coomassie blue, respectively. Wherein, MW represents a molecular weight marker, CL represents cell lysate after sonication, FT represents a flow-through streptavidin resin, and E represents an eluted protein;

FIG. 3A shows results of kinetic analysis for NAD⁺-dependent H₂ oxidation of ReSH in the presence or absence of O₂;

FIG. 3B shows results of kinetic analysis for Nicotinamide adenine dinucleotide (NADH)-dependent CO₂ reduction of RcFDH in the presence or absence of O₂;

FIGS. 4A and 4B show amounts of NADH and conversion of H₂ and CO₂ into formate over time by the NAD⁺-dependent ReSH and RcFDH cascade reactions;

FIG. 5 shows a HPLC calibration curve of formate;

FIGS. 6A to 5E show results of MALDI-TOF mass spectrometry for subunits of ReSH and RcFDH;

FIG. 7 shows concentrations of formic acid produced after 1 hour at different ratios of ReSH:RcFDH; and

FIG. 8 shows concentrations of H₂ and CO₂ converted into formate by the NAD⁺-dependent ReSH and RcFDH cascade reactions under 0%, and 5% O₂.

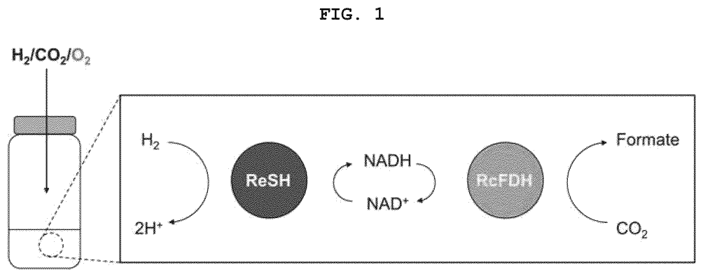

### DETAILED DESCRIPTION OF THE INVENTION

The present invention provides a process for producing formate using oxygen-tolerant enzymes.

The present invention provides a process for producing formate using oxygen-tolerant enzymes, which includes preparing a mixed enzyme by mixing hydrogenase with oxygen tolerance and formate dehydrogenase with oxygen tolerance; and mixing the mixed enzyme and a gas including H₂, CO₂ and NAD⁺, such that formate may be produced even in the presence of oxygen, and thereby utilizing hydrogen sources including oxygen, such as coke oven gas. That is, the present invention provides a process for producing formate, which is effective in solving the problem in that the production of formate was impossible due to the use of conventional hydrogenase and formate dehydrogenase, which are cheap and sustainable H₂ sources but contain O₂.

The present invention uses the mixed enzyme obtained by mixing hydrogenase with oxygen tolerance and formate dehydrogenase with oxygen tolerance. In the present invention, the oxygen tolerance means that the enzyme maintains activity without being deactivated even under a condition where oxygen is present.

The hydrogenase of the present invention refers to an enzyme that catalyzes the oxidation of hydrogen molecule (H₂). In the present invention, the oxidation of hydrogen through hydrogenase may be paired up with the reduction of an electron acceptor such as NAD⁺ or Nicotinamide adenine dinucleotide phosphate (NADP⁺), etc.

The hydrogenase of the present invention is not limited to those having a specific amino acid sequence or derived from a specific organism as long as they can catalyze the oxidation of hydrogen molecules while having oxygen tolerance.

In one embodiment, the hydrogenase of the present invention may be derived from aerobic microorganisms.

In one embodiment, the hydrogenase of the present invention may be derived from any one strain selected from the group consisting of Ralstonia eutropha, Escherichia coli and Aquifex aeolicus.

In one embodiment, the hydrogenase of the present invention may be [NiFe] H₂ase.

In one embodiment, the hydrogenase of the present invention may include a heterodimeric [NiFe] hydrogenase (HoxHY) subunit and a diaphorase (HoxFU) subunit.

In one embodiment, the hydrogenase of the present invention may include the amino acid sequence of any of SEQ ID NO: 1 to SEQ ID NO: 5.

In one embodiment, the hydrogenase with oxygen tolerance has activity under a condition where oxygen concentration is 10′% or less, 9% or less, 8%; or less, 7% or less, 6% or less, 5=t or less, 4% or less, 3 or less, 2 or less, 10 or less, 0.5 or less, or 0.1% or less.

In one embodiment, the oxygen tolerance of the hydrogenase of the present invention may be attributed to the reduction of O₂ bound to NiFe active site into either hydrogen peroxide or water.

Formate dehydrogenase of the present invention refers to an enzyme that catalyzes the reduction of carbon dioxide (CO₂) into formate. In the present invention, the reduction of carbon dioxide through formate dehydrogenase may be paired up with the oxidation of an electron donor such as NADH or Nicotinamide adenine dinucleotide phosphate (NADPH).

In the present invention, the formate dehydrogenase is not limited to those having a specific amino acid sequence or derived from a specific organism as long as they can catalyze the reduction of carbon dioxide into formate while having oxygen tolerance.

In one embodiment, the formate dehydrogenase of the present invention may be derived from aerobic microorganisms.

In one embodiment, the formate dehydrogenase of the present invention may be derived from any one strain selected from the group consisting of Rhodobacter capsulatus, Desulfovibrio vulgaris, Clostridium carboxidivorans and Methylobacterium extorquens.

In one embodiment, the formate dehydrogenase of the present invention may include the amino acid sequence of any of SEQ ID NO: 6 to SEQ ID NO: 8.

In one embodiment, the formate dehydrogenase of the present invention may include an FdsA subunit containing a bis(molybdopterin guanine dinucleotide) cofactor and an FdsGB diaphorase subunit.

In one embodiment, the formate dehydrogenase with oxygen tolerance has activity under a condition where oxygen concentration is 10%; or less, 9%; or less, 8% or less, 7% or less, 6% or less, 5% or less, 4% or less, 3 or less, 2.5% or less, 2% or less, 1.5% or less, 1% or less, 0.5% or less, 0.3% or less, or 0.1% or less.

In one embodiment, the oxygen tolerance of the formate dehydrogenase of the present invention may be attributed to the reduction of O₂ into hydrogen peroxide.

A mixing ratio of the hydrogenase with oxygen tolerance and the formate dehydrogenase with oxygen tolerance may be appropriately selected within a range where it is possible to produce formate, and is not limited to a specific ratio.

In one embodiment, the hydrogenase with oxygen tolerance and the formate dehydrogenase with oxygen tolerance may be mixed in a ratio of 0.1-50:1 (U/mL), such as 1-50:1, 5-50:1, 5-40:1, 5-30:1, 5-25:1, 5-20:1, 5-15:1, 10-40:1, 10-30:1, 10-20:1, 15-40:1, 15-35:1, 15-30:1, 15-25:1, 20-40:1 or 20-30:1.

The present invention uses a gas including H₂. The gas including H₂ of the present invention is not limited to a specific composition as long as it includes H₂, and is also not limited to one obtained from a specific source.

In one embodiment, the gas including H₂ may be obtained from any one source selected from the group consisting of byproduct hydrogen incidentally generated in processes such as a petrochemical or steel industry, gas derived from plastic or solid waste, cokes, naphtha, volcanic gas, mineral water, coal gas, solar heat, algae emissions, biomass, natural gas, fossil fuel, coal, peat, petroleum and natural gasoline.

In one embodiment, the gas including H₂ of the present invention may be coke oven gas produced from the steel industry.

In one embodiment, the gas including H₂ of the present invention may be byproduct hydrogen.

In one embodiment, the gas including H₂ of the present invention may be a gas derived from plastic or solid waste.

The gas including H₂ of the present invention may contain O₂. The gas including H₂ which contains O₂ of the present invention is not limited to a specific composition as long as it includes H₂ and O₂, and is also not limited to one obtained from a specific source.

In one embodiment, the gas including H₂ of the present invention may contain O₂ of 60% or less, 50% or less, 40% or less, 30% or less, 20% or less, 10% or less, 9% or less, 8% or less, 7% or less, 6%; or less, 5%; or less, 4% or less, 3% or less, 2% or less, 1% or less, 0.5% or less, or 0.1% or less based on a total weight of the gas.

### EXAMPLE

### 1. Materials

The 5× In-Fusion HD Enzyme Premix was purchased from Takara Bio (Kusatsu, Japan). Strep-Tactin XT 4 Flow high-capacity resin was obtained from IBA Life Sciences (Gottingen, Germany). Disposable PD-10 desalting columns were purchased from Cytiva (Marlborough, MA, USA). Vivaspin 6 centrifugal concentrators with a molecular weight cutoff (MWCO) of 100 kDa were purchased from Sartorius (Göttingen, Germany). A polypropylene column (1 mL) was purchased from Qiagen (Hilden, Germany). The Ziptip C₁₈ resin was purchased from Millipore (Burlington, MA, USA). All other chemical reagents were purchased from Sigma-Aldrich (St. Louis, MO, USA) unless otherwise stated.

### 2. Experimental Method

### 2.1 Construction of Plasmids and Strains

To construct the Strep-Tag II-fused Rhodobacter capsulatu-derived formate dehydrogenase (RcFDH) expression plasmid, pTrcHis-RcFDH was used as a template. Injection cloning was performed to substitute the hexahistidine-tag for strep tag II. pTrcHis-RcFDH was amplified by PCR with the in-fusion primer (FW: SEQ ID NO: 9, RV: SEQ ID NO: 10). The PCR product was mixed with 5× In-Fusion HD Enzyme Premix to generate pTrcHis-strep-RcFDH. The E. coli MC1061 strain was transformed with pTrcHis-Strep-RcFDH, and R. eutropha HF210 [pGE771] strain was used as the Ralstonia eutropha-derived hydrogenase (ReSH)-expressing strain.

### 2.2 Expression of ReSH AND RcFDH

For the expression of ReSH and RcFDH, a 7 L scale fermenter was used. First, a 10× H16 buffer (pH 7.0) containing 250 mM Na₂HPO₄ and 110 mM KH₂PO₄ was used as a medium. For 1 L of fructose-ammonium (FN) medium, 100 mL of 10× H16 buffer was mixed with 850 mL of sterile water (additional 13% (w/v) of Bacto agar in the case of solid agar plates) and autoclaved. Next, 10 mL of 20% (w/v) NH₄Cl, 1 mL of 20% (w/v) NH₄Cl, 1 mL of 20% (w/v) MgSO₄*7H₂O, 1 mL of 1% (w/v) CaCl₂*H₂O, 1 mL of 0.5% (w/v) FeCl₃*6H₂O (in 0.1 N HCl), 1 mM NiCl₂, and 1.25 mL of 40% (w/v) D-fructose were mixed and filled up to 1000 mL with sterile H₂O. A single colony of R. eutropha was pre-cultured in 50 mL of FN medium containing 10 μg mL⁻¹ tetracyclin until the OD at 436 nm reached 1. For the main culture, 5 L of modified fructose-glycerol-ammonium (FGN_{mod}) with 0.05% (w/v) glycerol, 5 mL of SL6 trace element solution, and 5 mL of 1 mM ZnCl₂ (added to the FN medium containing 10 μg/mL tetracycline) were prepared in the fermenter. The pre-culture was inoculated into the FGN_{mod} medium and subjected to 300 rpm shaking and 1 VVM aeration at 30° C. The pH range was maintained between 6.9 to 7.0 through automatic injection of 1 N NaOH. After 24 h, 5 mL of 1 mM NiCl₂ was added. When the OD at 436 nm reached 9-11, the cells were harvested by centrifugation at 6,000×g for 10 min, and stored at −80° C.

For RcFDH expression, a single-cell colony was pre-cultured in Luria-Bertani (LB) medium containing 150 μg mL⁻¹ ampicillin for 12 h at 37° C. For the main culture, 5 L of LB medium containing 150 μg mL⁻¹ ampicillin, 1 mM sodium molybdate, and 20 μM isopropyl β-D-1-thiogalactopyranoside was prepared in the fermenter. The pre-culture was inoculated into the LB medium and subjected to 100 rpm shaking and 0.1 VVM aeration at 30° C. After 24 h, the cells were harvested by centrifugation at 6,000×g for 10 min, and stored at −80° C.

### 2.3 Purification of ReSH and RcFDH

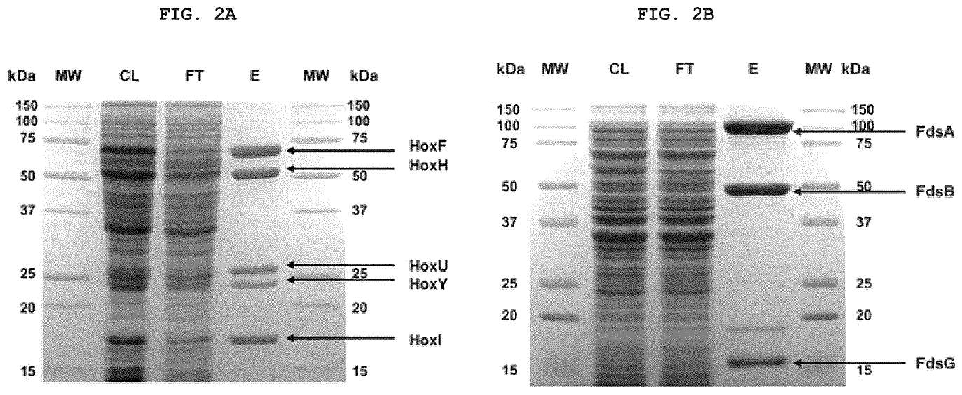

To purify ReSH and RcFDH, cell pellets were resuspended in 50 mM potassium phosphate buffer (pH 7.0) (Kpi buffer) containing 1 mg/mL lysozyme at a concentration of 1 g/10 mL. The resuspended cells were lysed by sonication (amplitude 28%, on/off 2 s/4 s) for 1 h. Insoluble cell debris was removed by centrifugation at 13,000×g for 30 min. Strep-Tactin XT 4 Flow high-capacity resin (2 mL) was mixed with the clear supernatants and incubated at 4° C. for 30 min. The resin was washed with Kpi buffer containing 300 mM potassium chloride on a gravity-flow polypropylene column to remove any impurities. The proteins were eluted with 3 mL of Kpi buffer containing 50 mM biotin, and buffer-exchanged with Kpi buffer containing 10 mM potassium nitrate using a PD-10 column. Protein purity was verified by SDS-PAGE (FIGS. 2A and 2B). The concentrations of the purified proteins were determined by measuring their absorbance at 280 nm using a microplate reader (Synergy, BioTek, Winooski, VT, USA). The extinction coefficients of ReSH and RcFDH were calculated to be 165, 710 and 350,000 M⁻¹, cm⁻¹, respectively, based on their amino acid sequences.

### 2.4 Matrix-Assisted Laser Desorption Ionization-Time of Flight (MALDI-TOF) Mass Spectrometry

Proteins in buffer were desalted using Ziptip C according to the manufacturer's protocol. The purified ReSH and RcFDH were mixed in a 1:1 (v/v) ratio with a sinapinic acid-saturated matrix solution consisting of 30% acetonitrile, 0.1% trifluoroacetic acid (TFA) and 70's water (v/v). The mixture was subjected to mass characterization by Autoflex speed (Bruker Corporation, Billerica, USA).

### 2.5 Enzyme Kinetics

The enzyme reaction kinetics of ReSH were measured for the NAD-dependent oxidation of H₂ to H⁺ in the presence or absence of O₂. A sealing cuvette was filled with 900 μL of Kpi buffer containing NAD⁺ and sealed; then, 100% H₂ and a mixed gas consisting of 10% O₂ and 90% N₂ (or 100% N₂ for anaerobic conditions) were injected simultaneously for 30 min at 10 mL/min. ReSH (2 mL, 80 nM) was purged with 10 mL/min N₂ gas bubbling in a 10 mL sealing vial for 30 min to remove O₂ from the air. The reaction was initiated by mixing 100 μL of 80 nM ReSH with a gas-saturated solution in the sealed cuvette. The final concentration of NAD⁺ was varied from 0 to 2 mM.

The enzyme reaction kinetics of RcFDH were measured for NADH-dependent reduction of CO₂ to formate in the presence or absence of O₂. The sealing cuvette was filled with 900 μL of Kpi buffer containing NADH and sealed; then, 100% CO₂ and a mixed gas consisting of 4% O₂ and 96 N₂ (or 100% N₂ for anaerobic conditions) were injected simultaneously for 30 min at 10 mL/min, respectively. RcFDH (2 mL, 2 μM) was purged with 10 mL/min N₂ gas bubbling in a 10 mL sealing vial for 30 min to remove O₂ from the air. The reaction was initiated by mixing 100 μL of 2 μM RcFDH with a gas-saturated solution in the sealing cuvette. The final concentration of NADH was varied from 0 to 1 mM.

All measurements were performed in triplicate based on the change in the absorbance at 365 nm in the cuvette, measured using a T60 UV-Vis spectrophotometer (PG Instruments Ltd, Lutterworth, UK). The change in the absorbance over 1 min was plotted using the Michaelis-Menten equation to calculate the kinetic parameters.

### 2.6 Formate Production and Quantification

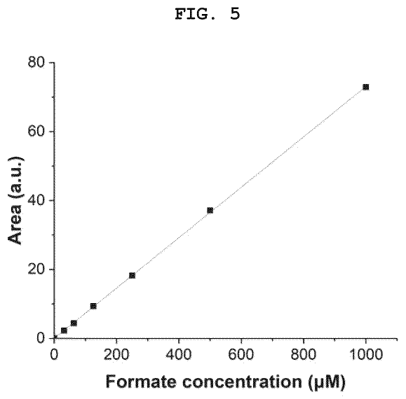

For the cascade reaction in the presence or absence of O₂, the gas content was controlled in a 20 mL polytetrafluoroethylene (PTFE) septa sealing vial. The vial was filled with 500 μL of reaction solution containing 3.2 U/mL ReSH, 0.16 U/mL RcFDH, 1 mM NAD⁺ and 0.5 M Kpi buffer, and then sealed. A needle was inserted into the septa for gas evacuation. Then, 10 mL/min CO₂ and 20 mL/min N₂/O₂ mixed gas were injected for 30 min (the needle did not enter the reaction solution). The O₂ ratios of the mixed gas varied from 0 to 2-4%; therefore, the final concentrations of O₂ were 0, 1 and 2%. The reaction was initiated by a 10 mL/min H₂ gas injection. Formate production was sampled every 20 min during incubation for 1 h, and 10 μL of 6 N H₂SO₄ was added to the 100 μL sample to inactivate the enzymes immediately. Additionally, 240 μL of distilled water was mixed with the sample, and the aggregate enzymes were removed by centrifugation at 13,000×g. Formate production was quantified by HPLC (1260, Agilent, CA, USA) equipped with a diode-array detector and an Aminex HPX-87H column (BIO-RAD, CA, USA) with a mobile phase of 5 μM H₂SO₄ at a flow rate of 0.6 mL/min. The retention time of formate was 13.010 min. The formate concentration was calculated using a formate calibration curve (FIG. 5).

### 3. Results

### 3.1 Preparation of ReSH and RcFDH

ReSH and RcFDH were expressed in R. eutropha and E. coli, respectively. These were purified using affinity resins, as described in the Materials and methods.

Five bands of purified ReSH subunits were observed, which matched with the expected molecular weights (HoxF, 68,110 Da; HoxH, 54,863 Da; HoxU, 26,173 Da; HoxY, 22,881 Da; HoxI, 18,567 Da) (FIG. 2A).

Three bands of purified RcFDH subunits were observed, which were consistent with the expected molecular weights (FdsA, 104,466 Da; FdsB, 52,699 Da; FdsG, 17,304 Da) (FIG. 2B).

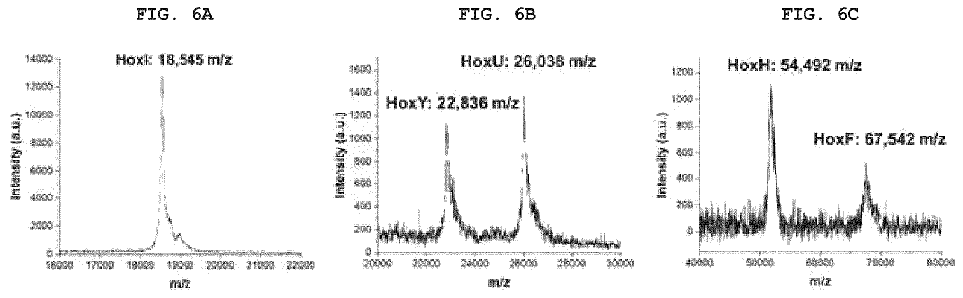

Both enzymes showed high purity. The identity of the purified enzymes was confirmed by MALDI-TOF mass spectrometry. The experimentally determined masses of ReSH subunits were 67,542, 54,492, 26,038, 22,836 and 18,545 m/z, which matched well with the expected masses (68,111, 54,864, 26,174, 22,882 and 18,568 m/z, respectively) with less than 1% deviation (FIGS. 6A, 6B and 6C). The experimentally determined masses of RcFDH subunits were 104,259, 52,385 and 17,136 m/z, which matched well with the expected masses (104,467, 52,700 and 17,305 m/z, respectively) with less than 1′% deviation (FIGS. 6D and 6E).

These results showed that the purified ReSH and RcFDH were successfully prepared.

### 3.2 Enzyme Kinetics in the Presence or Absence of O₂

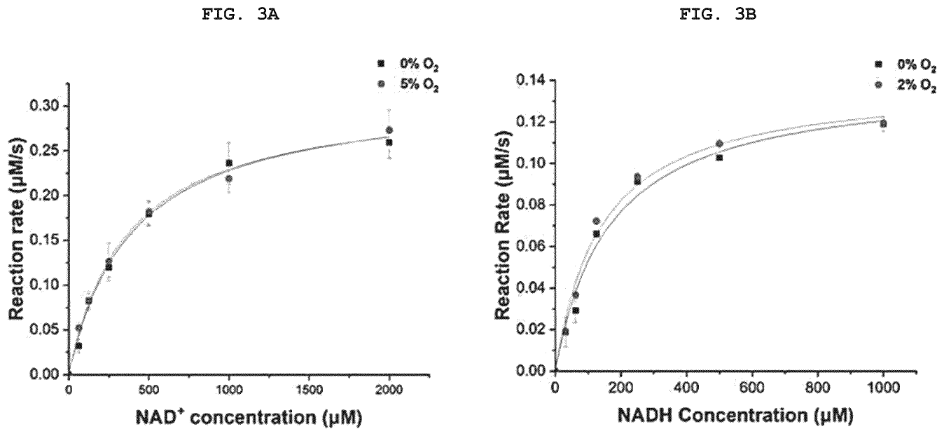

The present inventor investigated the enzymatic activities of ReSH and RcFDH in the presence or absence of O₂. The NAD⁺-dependent H₂ oxidation reaction rate by ReSH was measured, and the Michaelis-Menten curve was fitted to calculate the kinetic parameters using Origin 2022 program (FIG. 3A). Both k_{cat} and Kₘ values of ReSH showed an insignificant difference under the 0% and 5% O₂ conditions (Table 1). Similarly, the NADH-dependent CO₂ reduction reaction rate by RcFDH was measured, and the Michaelis-Menten curve was fitted to calculate the kinetic parameters (FIG. 3B). Likewise, k_{cat} and Kₘ values of RcFDH showed an insignificant difference between the 0% and 2% O₂ conditions (Table 2). These results show that the purified ReSH and RcFDH retained the enzymatic activity at least under less than 2% O₂.

TABLE 1

| O₂ concentration (%) | k_{cat}(s⁻¹) | Kₘ(mM)(NAD⁺) |
|---|---|---|
| 0 | 39.7 ± 1.5 | 0.393 ± 0.041 |
| 5 | 39.2 ± 1.3 | 0.364 ± 0.033 |

TABLE 2

| O₂ concentration (%) | k_{cat}(s⁻¹) | Kₘ(mM)(NADH) |
|---|---|---|
| 0 | 0.703 ± 0.043 | 0.166 ± 0.030 |
| 2 | 0.699 ± 0.035 | 0.141 ± 0.022 |

### 3.3 Cascade Reaction Condition Control

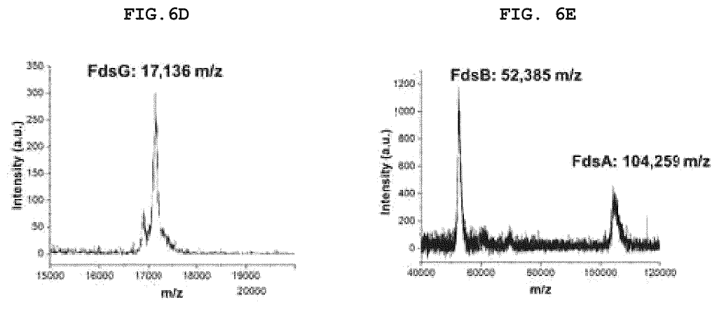

The present inventor determined the NAD⁺, ReSH and RcFDH contents for the cascade reaction of ReSH and RcFDH. Due to the relatively low k_{cat} value (Tables 1 and 2), the rate-determining step was the CO₂ reduction by RcFDH. Because the reaction rate of RcFDH was saturated at NADH concentrations above 1 mM (FIG. 3B), the NAD⁺ concentration was determined to be 1 mM. For the continuous CO₂ reduction by RcFDH, the concentration of ReSH was determined to maintain a state in which all NAD⁺ was reduced to NADH. The concentration of RcFDH was fixed at 0.08 U/mL, and the amount of ReSH was adjusted to 0, 0.08, 0.8 and 1.6 U/mL (U/mL ratio of ReSH:RcFDH=0:1, 1:1, 5:1, 10:1, 20:1). Reaction solutions were placed in a 20 mL sealing vial, and 10 mL/min CO₂ and 10 mL/min H₂ were injected for 1 h simultaneously, after which formate was measured (FIG. 7). Formate production was not observed in the reaction solution without ReSH. In contrast, substantial formate production was observed in the reaction solution with the three components (ReSH, RcFDH and NAD⁺). Formate production was saturated in a ratio of above 5:1. At higher ReSH concentrations, NAD⁺ was immediately converted to NADH through H₂ oxidation. Based on this result, the cascade reaction content was set to 1 mM NAD⁺, and the U/mL ratio of ReSH:RcFDH was set to 20:1.

### 3.4 Formate Production Under O₂ Conditions

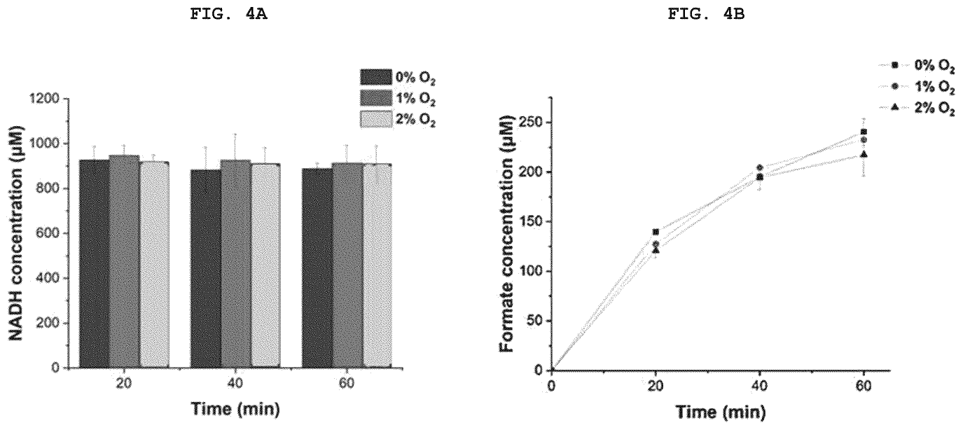

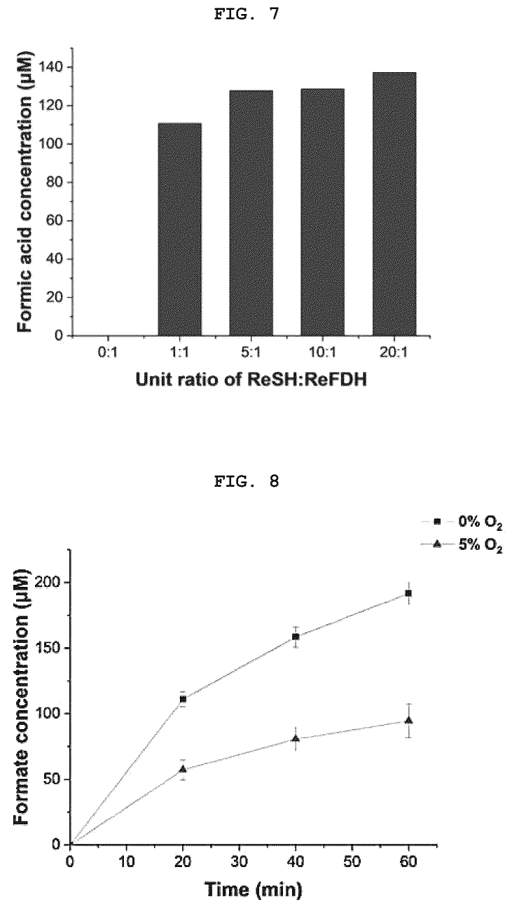

The present inventor demonstrated that H₂ and CO₂ were converted into formate under 0% to 2% O₂ conditions. ReSH, RcFDH and 1 mM NAD⁺ were mixed and placed in a 20 mL sealing vial. Changes in the concentrations of NADH and formate over time were investigated when O₂ (at a controlled concentration), H₂ and CO₂ were simultaneously and continuously injected into the vial. During the injection of the gases, under all 0; conditions between 0% and 2%, NAD⁺ was reduced to NADH and maintained at 1 mM by H₂ oxidation of ReSH (FIG. 4A). Furthermore, the formate concentration was increased continuously (FIG. 4B) due to the CO₂ reduction of RcFDH. Approximately 230 μM of formate was produced after 1 h, which showed a statistically insignificant difference at 0, 1 or 2% O₂ conditions (p>0.05). In order to investigate the O₂-tolerance limit of the system, the present inventor tested the formate production at a higher concentration of O₂ (FIG. 8). As compared to 0%, a substantial reduction in formate production at 5% O₂ was observed by the present inventor. Therefore, in the specific enzyme systems chosen by the present inventor, the O₂ tolerance limit was between 2 and 5%. The O₂-tolerance of both H₂ase and FDH is attributed to the reduction of O₂ bound to the active site of enzymes, leading to the reactivation of active site. Therefore, the present inventor speculated that the substantial loss of enzymatic activities at 5% O₂ results from that O₂ binding to the active site is more favorable than O₂ reduction at the active site. These results demonstrate, as hypothesized, the plausibility of a cascade reaction using ReSH and RcFDH, even in the presence of O₂.

### 4. Conclusions

The present inventor demonstrated the conversion of H₂ and CO₂ into formate using an NAD⁺-dependent cascade reaction of O₂-tolerant H₂ase and O₂-tolerant FDH in the presence of O₂.

### REFERENCE TO AN ELECTRONIC SEQUENCE LISTING

A sequence listing electronically submitted on Dec. 21, 2023 as a XML file named 20231221_LC0592321_TU_SEQ.XML, created on Dec. 21, 2023 and having a size of 13,037 bytes, is incorporated herein by reference in its entirety.

What is claimed is:

1. A process for producing formate, the process comprising:
preparing a mixed enzyme by mixing hydrogenase (H₂ase) with oxygen tolerance and formate dehydrogenase (FDH) with oxygen tolerance; and
mixing the mixed enzyme and a gas including H₂, CO₂ and Nicotinamide adenine dinucleotide phosphate (NAD⁺).

2. The process of claim 1, wherein the gas including H₂ contains O₂.

3. The process of claim 1, wherein the gas including H₂ is obtained from any one source selected from the group consisting of byproduct hydrogen incidentally generated in a process of a petrochemical or steel industry, gas derived from plastic or solid waste, coke, naphtha, volcanic gas, mineral water, coal gas, solar heat, algae emission, biomass, natural gas, fossil fuel, coal, peat, petroleum and natural gasoline.

4. The process of claim 1, wherein the hydrogenase is derived from any one strain selected from the group consisting of Ralstonia eutropha, Escherichia coli and Aquifex aeolicus.

5. The process of claim 1, wherein the formate dehydrogenase is derived from any one strain selected from the group consisting of Rhodobacter capsulatus, Desulfovibrio vulgaris, Clostridium carboxidivorans and Methylobacterium extorquens.

6. The process of claim 1, wherein the hydrogenase comprises the amino acid sequence of any of SEQ ID NO: 1 to SEQ ID NO: 5.

7. The process of claim 1, wherein the formate dehydrogenase comprises the amino acid sequence of any of SEQ ID NO: 6 to SEQ ID NO: 8.

8. The process of claim 2, wherein the gas including H2 contains 5% or less of O₂.

9. The process of claim 2, wherein the gas including H2 contains 2% or less of O₂.

10. The process of claim 1, wherein the hydrogenase and the formate dehydrogenase are mixed in a ratio of 5 to 25:1.


SEQUENCE LISTING (10 sequences)

SEQ ID NO: 1 - protein, 614 aa, Ralstonia sp.
MASWSHPQFEKGADSRITTILERYRSDRTRLIDILWDVQHEYGHIPDAVLPQLGAGLKLSPLDIRETASFYHFFLDKPSGKYRIYLCNSVIAKINGYQAVREALERETGIRFGETDPNGMFGLFDTPCIGLSDQEPAMLIDKVVFTRLRPGKITDIIAQLKQGRSPAEIANPAGLPSQDIAYVDAMVESNVRTKGPVFFRGRTDLRSLLDQCLLLKPEQVIETIVDSRLRGRGGAGFSTGLKWRLCRDAESEQKYVICNADEGEPGTFKDRVLLTRAPKKVFVGMVIAAYAIGCRKGIVYLRGEYFYLKDYLERQLQELREDGLLGRAIGGRAGFDFDIRIQMGAGAYICGDESALIESCEGKRGTPRVKPPFPVQQGYLGKPTSVNNVETFAAVSRIMEEGADWFRAMGTPDSAGTRLLSVAGDCSKPGIYEVEWGVTLNEVLAMVGARDARAVQISGPSGECVSVAKDGERKLAYEDLSCNGAFTIFNCKRDLLEIVRDHMQFFVEESCGICVPCRAGNVDLHRKVEWVIAGKACQKDLDDMVSWGALVRRTSRCGLGATSPKPILTTLEKFPEIYQNKLVRHEGPLLPSFDLDTALGGYEKALKDLEEVTR

SEQ ID NO: 2 - protein, 234 aa, Ralstonia sp.
MSIQITIDGKTLTTEEGRTLVDVAAENGVYIPTLCYLKDKPCLGTCRVCSVKVNGNVAAACTVRVSKGLNVEVNDPELVDMRKALVEFLFAEGNHNCPSCEKSGRCQLQAVGYEVDMMVSRFPYRFPVRVVDHASEKIWLERDRCIFCQRCVEFIRDKASGRKIFSISHRGPESRIEIDAELANAMPPEQVKEAVAICPVGTILEKRVGYDDPIGRRKYEIQSVRARALEGEDK

SEQ ID NO: 3 - protein, 209 aa, Ralstonia sp.
MRAPHKDEIASHELPATPMDPALAANREGKIKVATIGLCGCWGCTLSFLDMDERLLPLLEKVTLLRSSLTDIKRIPERCAIGFVEGGVSSEENIETLEHFRENCDILISVGACAVWGGVPAMRNVFELKDCLAEAYVNSATAVPGAKAVVPFHPDIPRITTKVYPCHEVVKMDYFIPGCPPDGDAIFKVLDDLVNGRPFDLPSSINRYD

SEQ ID NO: 4 - protein, 488 aa, Ralstonia sp.
MSRKLVIDPVTRIEGHGKVVVHLDDDNKVVDAKLHVVEFRGFEKFVQGHPFWEAPMFLQRICGICFVSHHLCGAKALDDMVGVGLKSGIHVTPTAEKMRRLGHYAQMLQSHTTAYFYLIVPEMLFGMDAPPAQRNVLGLIEANPDLVKRVVMLRKWGQEVIKAVFGKKMHGINSVPGGVNNNLSIAERDRFLNGEEGLLSVDQVIDYAQDGLRLFYDFHQKHRAQVDSFADVPALSMCLVGDDDNVDYYHGRLRIIDDDKHIVREFDYHDYLDHFSEAVEEWSYMKFPYLKELGREQGSVRVGPLGRMNVTKSLPTPLAQEALERFHAYTKGRTNNMTLHTNWARAIEILHAAEVVKELLHDPDLQKDQLVLTPPPNAWTGEGVGVVEAPRGTLLHHYRADERGNITFANLVVATTQNNQVMNRTVRSVAEDYLGGHGEITEGMMNAIEVGIRAYDPCLSCATHALGQMPLVVSVFDAAGRLIDERAR

SEQ ID NO: 5 - protein, 167 aa, Ralstonia sp.
MKEQEIDRIATMIYEAPLGEYIGRDGAAILAEHAAEARLLKGDEFLYRRGDVTSSFYIVTDGRLALVREKTNERTAPIVHVLEKGDLVGELGFIDQTPHSLSVRALGDAAVLSFSAESIKPLITEHPELIFNFMRAVIKRVHHVVVTVGEHERELQEYISTGGRGRG

SEQ ID NO: 6 - protein, 166 aa, Rhodobacter capsulatus
MGGSWSHPQFEKGMASMTDTARLRAILAAHRGREGALLPILHDVQAAFGFIPEDAYAPIAADLGLTRAEVAGVVGFYHDFRKAPAGRHVIKLCRAEACQAMGMDAVQARLESALGLRLGDSSEAVTLEAVYCLGLCACAPAAMVDDRLVGRLDAAAVAGIVAELGA

SEQ ID NO: 7 - protein, 500 aa, Rhodobacter capsulatus
MKIWLPCDAAAKACGAEAVLAALRLEAEKRGGALDIARNGSRGMIWLEPLLEVETPAGRIGFGPMTPADVPALFDALESHPKALGLVEEIPFFKRQTRLTFARCGRIEPLSLAQFAAAEGWAGLRKALKMTPAEVVEEVLASGLRGRGGAGFPTGIKWRTVAAAQADQKYIVCNVDEGDSGSFADRMLIEGDPFCLVEGMAIAGHAVGATRGYVYIRSEYPDAIAVMRAAIAMAKPFLAEAGFEMEVRVGAGAYVCGEETSLLNSLEGKRGTVRAKPPLPALKGLFGKPTVVNNLLSLAAVPWIIAHGAKAYESFGMDRSRGTIPLQIGGNVKRGGLFETGFGITLGELVEDICGGTASGRPVKAVQVGGPLGAYHPVSDYHLPFCYEQFAGQGGLVGHAGLVVHDDTADMLKLARFAMEFCAIESCGTCTPCRIGAVRGVEVIDRIAAGDASAMPLLDDLCQTMKLGSLCALGGFTPYPVQSAIRHFPADFPCAREAAE

SEQ ID NO: 8 - protein, 958 aa, Rhodobacter capsulatus
MKDLIIPPLDWTQDMGTPKREGAPVHLTIDGVEVTVPAGTSVLRAAAEAGISIPKLCATDNVEPVGSCRLCMVEIEGMRGTPTSCTTPVAPGMRVHTQTPQLQKLRRGVMELYISDHPLDCLTCAANGDCELQDMAGAVGLREVRYQAKDTHFARRDATGPNPRYIPKDNSNPYFSYDPAKCIVCMRCVRACEEVQGTFALTVMGRGFDARISPAAPDFLSSDCVSCGACVQACPTATLVEKSVERIGTPERKVVTTCAYCGVGCSFEAHMLGDQLVRMVPWKGGAANRGHSCVKGRFAYGYATHQDRILKPMIRDKITDPWREVNWTEALDFTATRLRALRDSHGADALGVITSSRCTNEETYLVQKLARAVFGTNNTDTCARVCHSPTGYGLKQTFGTSAGTQDFDSVEETDLALVIGANPTDGHPVFASRLRKRLRAGAKLIVVDPRRIDLLNTPHRGEAWHLQLKPGTNVAVMTAMAHVIVTEQIFDKRFIGDRCDWDEWADYAEFVANPEYAPEAVESLTGVPAGLLRQAARAYAAAPNAAIYYGLGVTEHSQGSTTVIAIANLAMMTGNIGRPGVGVNPLRGQNNVQGSCDMGSFPHEFPGYRHVSDDATRGLFERTWGVTLSSEPGLRIPNMLDAAVEGRFKALYVQGEDILQSDPDTRHVSAGLAAMDLVIVHDLFLNETANYAHVFLPGSTFLEKDGTFTNAERRINRVRRVMAPKAGFADWEVTQMLANALGAGWHYTHPSEIMAEIAATTPGFAAVTYEMLDARGSVQWPCNEKAPEGSPIMHVEGFVRGKGRFIRTAYLPTDEKTGPRFPLLLTTGRILSQYNVGAQTRRTENTVWHGEDRLEIHPTDAETRGIRDGDWVRLASRAGETTLRATVTDRVSPGVVYTTFHHPDTQANVVTTDTSDWATNCPEYKVTAVQVAASNGPSDWQQDYAAQAAAARRIEAAE

SEQ ID NO: 9 - DNA, 44 bp, synthetic construct
  source 1..44: mol_type = other DNA
gccacccgcagttcgaaaaaggtatggctagcatgacggatacc

SEQ ID NO: 10 - DNA, 44 bp, synthetic construct
  source 1..44: mol_type = other DNA
cgaactgcgggtggctccaagaaccccccatggtttattcctcc